(19) United States
(12) Patent Application Publication
(10) Pub. No.: US 2013/0096145 A1
(43) Pub. Date: Apr. 18, 2013

(54) Use of 1H-quinazoline-2,4-diones

(71) Applicant: Hans O. Kalkman, Basel (CH)

(72) Inventor: Hans O. Kalkman, Basel (CH)

(73) Assignee: Novartis AG, Basel (CH)

(21) Appl. No.: 13/805,200

(22) PCT Filed: Jun. 24, 2011
(86) PCT No.: PCT/EP2011/060634

§ 371 Date: Dec. 18, 2012

(30) Foreign Application Priority Data
Jun. 24, 2010 (EP) 101672533

(51) Int. Cl. C07D 405/10; C07D 403/10; C07D 239/96

(52) U.S. Cl. CPC C07D 405/10; C07D 239/96; C07D 403/10
USPC 514/266.24; 544/285; 514/266.3; 544/284

(57) ABSTRACT

The invention concerns the use of competitive AMPA receptor antagonists for the treatment, prevention or delay of progression of spasticity.

### FIELD OF THE INVENTION

The present invention relates to new pharmaceutical uses of 1H-quinazoline-2,4-diones, their pharmaceutically acceptable salts, and prodrugs thereof specifically for the treatment of spasticity and related conditions and as a muscle relaxant.

### BACKGROUND OF THE INVENTION

Spasticity is a disorder involving constant, involuntary contraction of one or more muscle groups. Several pathological conditions can lead to spasticity, such as e.g. ischemic or traumatic spinal cord injury, brain trauma, multiple sclerosis, cerebral palsy or Parkinson disease. These conditions have as a common denominator an increased peripheral muscle tone caused by an enhanced α-motoneuron activity. Although the pathophysiologic basis of spasticity is not fully understood, commonly proposed mechanisms for this exaggerated motoneuron activity include the following: 1) increased primary afferent activity, 2) loss of descending inhibition, 3) loss of segmental inhibitory interneurons. The changes in muscle tone probably result from alterations in the balance of inputs from reticulospinal and other descending pathways to the motor and interneuronal circuits of the spinal cord, and the absence of an intact corticospinal system. Loss of descending tonic or phasic excitatory and inhibitory inputs to the spinal motor apparatus, alterations in the segmental balance of excitatory and inhibitory control, denervation supersensitivity, and neuronal sprouting may be observed. Once spasticity is established, the chronically shortened muscle may develop physical changes such as shortening and contracture that further contribute to muscle stiffness. Presently available treatments for spasticity include e.g. physical and occupational therapy, oral medications, intrathecal baclofen therapy ITB, chemodenervation and surgical treatments. Although useful in some cases, these methods are not universally successful in patient treatment. For example, the degree of improvement in patient symptoms or discomfort may be modest or accompanied by debilitating side effects. Alternatively and/or in addition, the treatment may be painful, invasive, involve a long recovery time or be otherwise traumatic for the patient.

A brief summary of some presently available oral medication treatments follows below.

### Oral Medications

Examples of some presently available oral medications include Baclofen (Lioresal®), Benzodiazepines (Valium® and Klonopin®), Dantrolene sodium (Dantrium®), Imidazolines (Clonidine and Tizanidine), Gabapentin Fampridine® and botulinum toxin. It is often necessary for efficacy to use these medications in combination either with each other or with other techniques, with resultant decrease in patient convenience. In addition, some or all of these drugs have been found to give rise to numerous side effects in patients e.g. drowsiness or sedation, weakness, diarrhea, decreased muscle tone, confusion, fatigue, nausea, dizziness or balance problems, liver problems, increased difficulty in controlling seizures in epilepsy patients, increased blood pressure-lowering effects of other medications, seizures, dry mouth, addiction, hallucinations, cognition impairment, memory impairment, rebound spasticity, clumsiness, behavioral problems, loss of strength and additive effects with alcohol or other CNS depressants, risk of drug abuse.

The efficacy of these medications varies from patient to patient as does the type and severity of the side effects. There is therefore a need for alternative or improved agents for the treatment and amelioration of spasticity, that do not give rise to some or all of the above disadvantages of traditional spasticity medications.

In more recent theories of spasticity, activated spinal astrocytes and microglia are thought to contribute to motoneuron hyper-excitation. Hypoxia or traumatic cell injury in the brain and spinal cord, or the auto-immune process of multiple sclerosis activates spinal astrocytes and microglia. Glia cells play a key role in sustaining low glutamate levels by an effective uptake system; however the expression of glutamate transporters is reduced in activated glia (Hu et al, 2000, Neuroimmunomodulation 7, 153-159). Activation of AMPA receptors in astrocytes causes release of glutamate, thus leading to a positive feedback process between motoneurons and glia (reviewed by De Leo et al, 2006, Pain 122, 17-21). Ischemic paraplegia in rats led to a specific increase in expression of iGluR1 AMPA receptors in spinal cord astrocytes. Selective downregulation of this AMPA receptor by means of intrathecal application of antisense-RNA, resulted in a potent reduction of spasticity and rigidity. Tezamapanel, which is a competitive AMPA antagonist and which appears to produce suppression of spasticity Hefferan et al, 2007, J Neurosci 27, 11179-11191), has to be administered to the patients via intrathecal injection. Unfortunately, none of the current competitive AMPA antagonists, including tezampanel, is orally bio-available. It is well understood that properties required for high affinity at the AMPA receptor are contrary to those required for oral bioavailability. Therefore there is a continued need to develop therapeutic agents for the treatment of spasticity with a further improved pharmacokinetic profile whilst at the same time achieving a good potency and safety profile. In particular, the provision of medicinal agents for the treatment of spasticity with enhanced bioavailability is of therapeutic advantage. Oral bioavailability is an important factor limiting the therapeutic applications of bioactive compounds. It would be thus advantageous to provide therapeutic agents for the treatment of spasticity with enhanced bioavailability.

### SUMMARY OF THE INVENTION OF THE INVENTION

A first aspect of the invention relates to a compound, 1H-quinazoline-2,4-diones of formula (I)

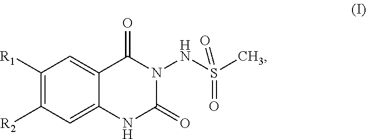

wherein

R₁ is C₁-C₅alkyl substituted by one, two or three substituents selected from hydroxy, C₁-C₆alkoxy or C₅-C₆cycloalkoxy; C₅-C₆cycloalkyl substituted by one, two or three substituents selected from hydroxy, C₁-C₆alkoxy or C₅-C₆cydoalkoxy; or R₁ is

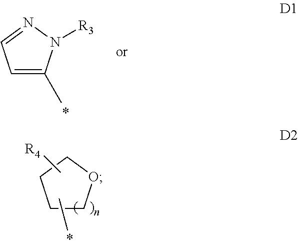

R₃ is C₁-C₆alkyl, hydroxy or C₁-C₆alkoxy-C₁-C₆alkyl;

R₄ is hydrogen or C₁-C₆alkyl;

n is 1 or 2;

R₂ is C₁-C₃alkyl or C₁-C₃fluoroalkyl;

and their pharmaceutically acceptable salts;

for use in a method for the treatment, prevention or delay of progression of spasticity.

A second aspect of the invention relates to a pharmaceutical composition comprising a 1H-quinazoline-2,4-dione of formula (I) for use in a method of treatment, prevention or delay of progression of spasticity.

A third aspect of the invention relates to the use of a 1H-quinazoline-2,4-dione of formula (I) for the manufacture of a medicament for the treatment, prevention or delay of progression of spasticity.

A fourth aspect of the invention relates to a method for the treatment, prevention or delay of progression of spasticity in a subject in need of such treatment, which comprises administering to said subject a therapeutically effective amount of a 1H-quinazoline-2,4-dione of formula (I).

A fifth aspect of the invention concerns the use of a 1H-quinazoline-2,4-dione of formula (I) for the treatment (whether therapeutic or prophylactic), prevention or delay of progression of spasticity.

A sixth aspect of the invention relates to a 1H-quinazoline-2,4-dione of formula (I) for the treatment, prevention or delay of progression of spasticity.

### DETAILED DESCRIPTION OF THE INVENTION

The invention relates to a compound, 1H-quinazoline-2,4-diones of formula (I)

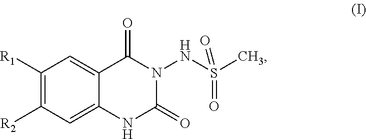

wherein

R₁ is C₁-C₆alkyl substituted by one, two or three substituents selected from hydroxy, C₁-C₆alkoxy or C₅-C₆cycloalkoxy; C₅-C₆cycloalkyl substituted by one, two or three substituents selected from hydroxy, C₁-C₆alkoxy or C₅-C₆cycloalkoxy; or

R₁ is

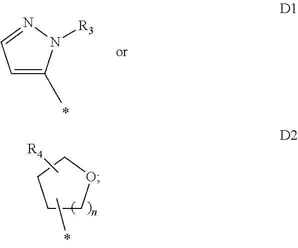

R₃ is C₁-C₆alkyl, hydroxy or C₁-C₆alkoxy-C₁-C₆alkyl;

R₄ is hydrogen or C₁-C₆alkyl;

n is 1 or 2;

R₂ is C₁-C₃alkyl or C₁-C₃fluoroalkyl;

their pharmaceutically acceptable salts, and their prodrugs thereof;

for use in a method for the treatment, prevention or delay of progression of spasticity.

The compound of formula (I) is a competitive AMPA antagonist. It is well understood that allosteric (non-competitive) antagonists provide an insurmountable blockade of AMPA receptors, potentially preventing any AMPA receptor-mediated neurotransmission at the synapse. In contrast, a high concentration of glutamate at the synapse can still activate the post-synaptic membrane in the presence of a competitive AMPA antagonist (albeit with a lower efficacy). Competitive AMPA antagonists may therefore exhibit an improved safety profile, as they will not fully block neurotransmission, but instead reduce the exaggerated glutamate signaling observed in some neurological disease, e.g. epilepsy.

Compounds of the formula (I) not only block AMPA-induced glutamate release from activated astrocytes but after oral dosing also suppress spasticity.

The compound of the invention of formula (I) in addition to the advantage of being a competitive AMPA antagonist receptor inhibitor, presents also the advantage of being a selective competitive AMPA antagonist. Furthermore the compound of the invention of formula (I) is capable of penetrating the blood brain barrier and may be formulated in an oral dosage form.

In the present specification, the following definitions shall apply if no specific other definition is given:

Bonds with the asterisk (*) denote point of binding to the rest of the molecule.

The term “treatment” is intended to mean administration or application of the medicament containing 1H-quinazoline-2,4-diones to a patient affected by spasticity and related conditions.

The term “prevention” is intended to mean administration or application of the medicament containing 1H-quinazoline-2,4-diones to a patient in order to prevent the onset of spasticity and related conditions, e.g. administration or application of the medicament shortly after a spinal cord injury.

The term “delay of progression” is intended to mean administration or application of the medicament containing 1H-quinazoline-2,4-diones to a patient in order to postpone the progression spasticity and related conditions.

“C₁-C₆alkyl ” represents a straight-chain or branched-chain alkyl group; for example, methyl, ethyl, n- or iso-propyl, n-, iso-, sec- or tert-butyl, n-pentyl, n-hexyl, with particular preference given to methyl, ethyl, n-propyl and iso-propyl.

“C₅-C₆cycloalkyl” represents cyclopentyl or cyclohexyl; preferably cyclopentyl.

Each alkyl/cycloalkyl-part of “alkoxy”, “cycloalkoxy”, “alkoxyalkyl” and “fluoroalkyl” shall have the same meaning as described in the above-mentioned definitions of “alkyl”/“cycloalkyl”.

“C₁-C₃fluoroalkyl” preferably represents trifluoromethyl, difluoromethyl or fluoromethyl.

It will be understood that any discussion of methods or references to the active ingredients includes said active ingredient in free form and in form of a pharmaceutically acceptable salt. If the active ingredients have, for example, at least one basic center, they can form acid addition salts. If the active ingredients have, for example, at least one acidic center (for example COOH) they can form salts with bases. The active ingredient or a pharmaceutically acceptable salt thereof may also be used in the form of a hydrate or may include other solvents used for crystallization.

A “pharmaceutically acceptable salt” is intended to mean a salt of a free base/free acid of a compound represented by formula (I) that is not toxic, biologically intolerable, or otherwise biologically undesirable. Preferred pharmaceutically acceptable salts are those that are pharmacologically effective and suitable for contact with the tissues of patients without undue toxicity, irritation, or allergic response. Such salts are known in the field (e.g. S. M. Berge, et al, “Pharmaceutical Salts”, J. Pharm. Sd., 1977, 66:1-19; and “Handbook of Pharmaceutical Salts, Properties, Selection, and Use”, Stahl, R H., Wermuth, C. G., Eds.; Wiley-VCH and VHCA: Zurich, 2002).

In one embodiment of the invention, the 1H-quinazoline-2,4-diones of formula (I) is used in free form. The 1H-quinazoline-2,4-diones of formula (I) and their manufacture are known from WO 2006/108591 or can be prepared analogously to said reference. WO 2006/108591 is incorporated herein by reference.

On account of asymmetrical carbon atom(s) that may be present in the 1H-quinazoline-2,4-diones of formula (I), their pharmaceutically acceptable salts and prodrugs thereof, the compounds may exist in optically active form or in form of mixtures of optical isomers, e.g. in form of racemic mixtures or diastereomeric mixtures. All optical isomers and their mixtures, including racemic mixtures, are part of the present invention.

In one embodiment of the invention, the 1H-quinazoline-2,4-dione of formula (I), its pharmaceutically acceptable salts and prodrugs thereof is a compound, wherein R₁ is C₁-C₆alkyl substituted by one two or three substituents selected from hydroxy, C₁-C₆alkoxy or C₅-C₆cycIoalkoxy; and R₂ is C₁-C₃alkyl or C₁-C₃fluoroalkyl.

In one embodiment of the invention, the 1H-quinazoline-2,4-dione of formula (I), its pharmaceutically acceptable salts and prodrugs thereof is a compound, wherein R₁ is

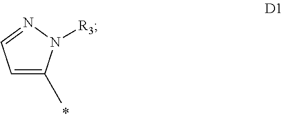

R₃ is C₁-C₆alkyl, hydroxy or C₁-C₆alkoxy-C₁-C₆alkyl; and R₂ is C₁-C₃alkyl or C₁-C₃fluoroalkyl.

In one embodiment of the invention, the 1H-quinazoline-2,4-dione of formula (I), its pharmaceutically acceptable salts and prodrugs thereof is a compound, wherein R₁ is

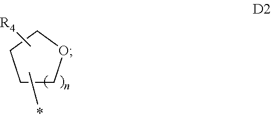

R₄ is hydrogen or C₁-C₆alkyl; n is 1 or 2; and R₂ is C₁-C₃alkyl or C₁-C₃fluoroalkyl.

In one embodiment of the invention, the 1H-quinazoline-2,4-dione of formula (I) is a compound selected from the group consisting of

A-1: N-[6-(1-Hydroxy-ethyl)-2,4-dioxo-7-trifluoromethyl-1,4-dihydro-2H-quinazolin-3-yl]-methanesulfonamide;

A-2: N-[6-(1-Methoxy-ethyl)-2,4-dioxo-7-trifluoromethyl-1,4-dihydro-2H-quinazolin-3-yl]-methanesulfonamide;

A-3: N-[6-(1-Hydroxy-propyl)-2,4-dioxo-7-trifluoromethyl-1,4-dihydro-2H-quinazolin-3-yl]-methanesulfonamide;

A-4: N-[6-(1-Isopropoxy-ethyl)-2,4-dioxo-7-trifluoromethyl-1,4-dihydro-2H-quinazolin-3-yl]-methanesulfonamide;

A-5: N-[6-(1-Ethoxy-ethyl)-2,4-dioxo-7-trifluoromethyl-1,4-dihydro-2H-quinazolin-3-yl]-methanesulfonamide;

A-6: N-[2,4-Dioxo-6-(1-propoxy-propyl)-7-trifluoromethyl-1,4-dihydro-2H-quinazolin-3-yl]-methanesulfonamide;

A-7: N-[6-(1-isopropoxy-propyl)-2,4-dioxo-7-trifluoromethyl-1,4-dihydro-2H-quinazolin-3-yl]-methanesulfonamide;

A-8: N-[7-Difluoromethyl-6-(1-ethoxy-ethyl)-2,4-dioxo-1,4-dihydro-2H-quinazolin-3-yl]-methanesulfonamide;

A-9: N-[2,4-Dioxo-6-(1-propoxy-ethyl)-7-trifluoromethyl-1,4-dihydro-2H-quinazolin-3-yl]-methanesulfonamide;

A-10: N-[6-(1-Butoxy-ethyl)-2,4-dioxo-7-trifluoromethyl-1,4-dihydro-2H-quinazolin-3-yl]-methanesulfonamide;

A-11: N-[6-(1-Isobutoxy-ethyl)-2,4-dioxo-7-trifluoromethyl-1,4-dihydro-2H-quinazolin-3-yl]-methanesulfonamide;

A-12: N-[6-(1-methoxy-butyl)-2,4-dioxo-7-trifluoromethyl-1,4-dihydro-2H-quinazolin-3-yl]-methanesulfonamide;

A-13: N-[6-(1-Ethoxy-propyl)-2,4-dioxo-7-trifluoromethyl-1,4-dihydro-2H-quinazolin-3-yl]-methanesulfonamide;

A-14: N-[6-(1-Cyclopentyloxy-ethyl)-2,4-dioxo-7-trifluoromethyl-1,4-dihydro-2H-quinazolin-3-yl]-methanesulfonamide;

A-15: N-[6-(1-Hydroxy-butyl)-2,4-dioxo-7-trifluoromethyl-1,4-dihydro-2H-quinazolin-3-yl]-methanesulfonamide;

A-16: N-[6-(1-Methoxy-2-methyl-propyl)-2,4-dioxo-7-trifluoromethyl-1,4-dihydro-2H-quinazolin-3-yl]-methanesulfonamide;

A-17: N-[6-(3-Hydroxy-propyl)-2,4-dioxo-7-trifluoromethyl-1,4-dihydro-2H-quinazolin-3-yl]-methanesulfonamide;

A-18: N-[6-(1-Hydroxy-3-methoxy-propyl)-2,4-dioxo-7-trifluoromethyl-1,4-dihydro-2H-quinazolin-3-yl]-methanesulfonamide;

A-19: N-[6-(1-Hydroxy-2-methyl-propyl)-2,4-dioxo-7-trifluoromethyl-1,4-dihydro-2H-quinazolin-3-yl]-methanesulfonamide;

B-1: N-[2,4-Dioxo-6-(tetrahydro-pyran-2-yl)-7-trifluoromethyl-1,4-dihydro-2H-quinazolin-3-yl]-methanesulfonamide;

B-2: N-[2,4-Dioxo-6-(tetrahydro-furan-2-yl)-7-trifluoromethyl-1,4-dihydro-2H-quinazolin-3-yl]-methanesulfonamide;

B-3: N-[2,4-Dioxo-6-(tetrahydro-furan-3-yl)-7-trifluoromethyl-1,4-dihydro-2H-quinazolin-3-yl]-methanesulfonamide;

C-1: N-{7-Isopropyl-6-[2-(2-methoxy-ethyl)-2H-pyrazol-3-yl]-2,4-dioxo-1,4-dihydro-2H-quinazolin-3-yl}-methanesulfonamide;

C-2: N-[6-(2-Isopropyl-2H-pyrazol-3-yl)-2,4-dioxo-7-trifluoromethyl-1,4-dihydro-2H-quinazolin-3-yl]-methanesulfonamide;

C-3: N-[7-Fluoromethyl-6-(2-isopropyl-2H-pyrazol-3-yl)-2,4-dioxo-1,4-dihydro-2H-quinazolin-3-yl]-methanesulfonamide;

C-4: N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;

C-5: N-[6-(2-Hydroxy-2H-pyrazol-3-yl)-2,4-dioxo-7-trifluoromethyl-1,4-dihydro-2H-quinazolin-3-yl]-methanesulfonamide;

C-6: N-[7-Ethyl-6-(2-isopropyl-2H-pyrazol-3-yl)-2,4-dioxo-1,4-dihydro-2H-quinazolin-3-yl]-methanesulfonamide;

C-7: N-[7-Isopropyl-6-(2-methyl-2H-pyrazol-3-yl)-2,4-dioxo-1,4-dihydro-2H-quinazolin-3-yl]-methanesulfonamide;

C-8: N-[7-Isopropyl-6-(2-isopropyl-2H-pyrazol-3-yl)-2,4-dioxo-1,4-dihydro-2H-quinazolin-3-yl]-methanesulfonamide;

C-9: N-[7-Difluoromethyl-6-(2-methyl-2H-pyrazol-3-yl)-2,4-dioxo-1,4-dihydro-2H-quinazolin-3-yl]-methanesulfonamide;

C-10: N-[7-Difluoromethyl-6-(2-isopropyl-2H-pyrazol-3-yl)-2,4-dioxo-1,4-dihydro-2H-quinazolin-3-yl]-methanesulfonamide;

C-11: N-[7-Ethyl-6-(2-methyl-2H-pyrazol-3-yl)-2,4-dioxo-1,4-dihydro-2H-quinazolin-3-yl]-methanesulfonamide;

C-12: N-[7-Ethyl-6-(2-ethyl-2H-pyrazol-3-yl)-2,4-dioxo-1,4-dihydro-2H-quinazolin-3-yl]-methanesulfonamide;

C-13: N-[7-Fluoromethyl-6-(2-methyl-2H-pyrazol-3-yl)-2,4-dioxo-1,4-dihydro-2H-quinazolin-3-yl]-methanesulfonamide;

C-14: N-[7-(1-fluoro-ethyl)-6-(2-methyl-2H-pyrazol-3-yl)-2,4-dioxo-1,4-dihydro-2H-quinazolin-3-yl]-methanesulfonamide;

C-15: N-[7-(1,1-difluoro-ethyl)-6-(2-methyl-2H-pyrazol-3-yl)-2,4-dioxo-1,4-dihydro-2H-quinazolin-3-yl]-methanesulfonamide;

C-16: N-[7-(1,1-difluoro-ethyl)-6-(2-isopropyl-2H-pyrazol-3-yl)-2,4-dioxo-1,4-dihydro-2H-quinazolin-3-yl]-methanesulfonamide;

C-17: N-[7-(1-fluoro-ethyl)-6-(2-isopropyl-2H-pyrazol-3-yl)-2,4-dioxo-1,4-dihydro-2H-quinazolin-3-yl]-methanesulfonamide; and

C-18: N-[6-(2-Methyl-2H-pyrazol-3-yl)-2,4-dioxo-7-trifluoromethyl-1,4-dihydro-2H-quinazolin-3-yl]-methanesulfonamide.

The compounds of the invention, including the specific exemplified compounds, may be prepared by any suitable method, e.g. as described in WO 2006/108591.

In one embodiment of the invention, the 1H-quinazoline-2,4-dione of formula (I) is a compound selected from the group consisting of compound A-1, A-2, A-3, A-4, A-5, A-6, A-7, A-8, A-9, A-10, A-11, A-12, A-13, A-14, A-15, A16, A17, A-18 and A-19.

In one embodiment of the invention, the 1H-quinazoline-2,4-dione of formula (I) is a compound selected from the group consisting of compound B-1, B-2 and B-3.

In one embodiment of the invention, the 1H-quinazoline-2,4-dione of formula (I) is a compound selected from the group consisting of compound C-1, C-2, C-3, C-4, C-5, C-6, C-7, C-8, C-9, C-10, C-11, C-12, C-13, C-14, C-15, C-16, C-17 and C-18.

Advantageous compounds of the invention, i.e., the 1H-quinazoline-2,4-diones of formula (I), should be well absorbed from the gastrointestinal tract, penetrate the blood brain barrier, be sufficiently metabolically stable and possess favorable pharmacokinetic properties.

Preferred compounds, having superior bioavailibility are 1H-quinazoline-2,4-dione of formula (I) selected from the group consisting of compounds: A-1, A-2, A-3, A-4, A-5, A-6, A-7, A-13, A-14, A-15, A-18, B-2, B-3, C-1, C-2, C-3, C-4, C-5, C-6, C-7, C-8, C-9, C-10, C-11, C-12, C-15, C-16, C-17 and C-18.

More preferred compounds, having superior bioavailibility are 1H-quinazoline-2,4-dione of formula (I) selected from the group consisting of compounds: A-1, A-2, A-3, A-4, A-5, A-7, A-15, B-2, B-3, C-1, C-2, C-3, C-6, C-7, C-8, C-9, C-10, C-11, C-12, C-15, C-17 and C-18.

Further more preferred compounds, having superior bioavailibility are 1H-quinazoline-2,4-dione of formula (I) selected from the group consisting of compounds: A-2, A-3, A-4, A-5 B-2, C-2, C-3,C-7, C-9, C-10, C-11, C-15 and C-18.

Most preferred compounds, having superior bioavailibility are 1H-quinazoline-2,4-dione of formula (I) selected from the group consisting of compounds: A-2, A-5, B-2, C-7, C-9 and C-11.

Compounds for use in the present invention are either obtained in the free form, as a salt thereof, or as prodrug derivatives thereof.

The term “prodrug” as used herein relates to a compound, which converts in vivo into a compound used in the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. The term “prodrug,” as used herein, represents in particular compounds which are transformed in vivo to the parent compound, for example, by hydrolysis in blood, for example as described in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), and “The Organic Chemistry of Drug Design and Drug Action”, 2^{nd} Edition, R B Silverman (particularly Chapter 8, pages 497 to 557), Elsevier Academic Press, 2004.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. alkyl esters |
| Alcohol | Esters, including e.g. sulfates and phosphates as |
| | well as carboxylic acid esters |
| Amine | Amides, carbamates, imines, enamines, |
| Carbonyl (aldehyde, | Imines, oximes, acetals/ketals, enol |
| ketone) | esters, oxazolidines and thiazoxolidines |

Prodrugs also include compounds convertible to the active drug by an oxidative or reductive reaction. As examples may be mentioned:

### Oxidative Activation

N- and O-dealkylation
Oxidative deamination
N-oxidation
Epoxidation

### Reductive Activation

Azo reduction
Sulfoxide reduction
Disulfide reduction
Bioreductive alkylation
Nitro reduction.

Each of the above described reactions and/or reaction steps can be used individually or in combination in a method to prepare a AMPA-inhibitor or a prodrug thereof.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

Preferred prodrugs of the invention should be well absorbed from the gastrointestinal tract, be transformed into the parent compound (or active principle, being the compound that in-vivo acts as AMPA receptor antagonist), the parent compound should be sufficiently metabolically stable and possess favorable pharmacokinetic properties.

Further preferred prodrugs of the invention lead to an oral bioavailability of the parent compound which is comparable to the bioavailability when administered as a drug. Further preferred prodrugs of the invention exhibit increased oral bioavailability compared to the parent compound when administered as a drug. Oral bioavailability may manifest itself in different ways: (i) a biological effect may be achieved after oral administration when the parent compound is less effective upon oral administration, (ii) an earlier onset of action upon oral administration, (iii) a lower dose needed to achieve the same effect, (iv) a higher effect achieved by the same dose or (v) a prolonged action at the same dose.

Further preferred prodrugs of the invention are transformed into parent compounds which in-vivo bind potently to AMPA receptors whilst showing little affinity for other receptors.

Further prodrugs of the invention—when the active principle is targeted against receptors in the central nervous system—are transformed into parent compounds that cross the blood brain barrier freely.

Further prodrugs of the invention—when the active principle is targeted selectively against receptors in the peripheral nervous system—are transformed into parent compounds that do not cross the blood brain barrier.

Prodrugs, parent compounds and released pro-moieties should be non-toxic and demonstrate few side-effects.

Furthermore, the ideal prodrug of the invention will be able to exist in a physical form that is stable, non-hygroscopic and easily formulated.

The higher oral bioavailability of the compounds for use in the invention may give rise to the following beneficial effects relating to less bioavailable compounds: (i) an enhanced biological effect may be achieved after oral administration; (ii) an earlier onset of action may be observed following oral administration; (iii) a lower dose may be needed to achieve the same effect; (iv) a higher effect may be achieved by the same dose or (v) a prolonged action may be observed at the same dose.

Preferably the compound for use in the invention when tested in-vivo potently binds to AMPA receptors whilst showing little affinity for other receptors.

In the present specification, the following definitions shall apply if no specific other definition is given:

The term “spasticity” includes spasticity as an isolated condition or spasticity associated with further conditions e.g. epilepsy, MS, cerebral palsy, spinal cord injury, acquired brain injury, including stroke and no neurological disease, such as cancer. For example spasticity includes spasticity associated with MS.

The term “subject” as used herein refers to a human or non-human being, preferably a human, especially to a patient being diagnosed with spasticity.

The term “treatment” as used herein refers to any type of treatment that imparts a benefit to a subject affected with a disease, e.g. a patient diagnosed with a disease, including improvement in the condition of the subject (e.g. in one or more symptoms), delay in the progression of the disease etc. Treatment typically comprises a reduction in the symptoms associated with spasticity.

The term “prevention” is intended to mean administration or application of the medicament containing 1H-quinazoline-2,4-diones to a patient in order to prevent the onset of spasticity and related conditions, e.g. administration or application of the medicament shortly after a spinal cord injury.

The term “delay of progression” is intended to mean administration or application of the medicament containing 1H-quinazoline-2,4-diones to a patient in order to postpone the progression spasticity and related conditions.

Spasticity is experienced in different degrees, muscles and severity by different people. Severity of spasticity may be measured using various means, e.g., subjective reported outcome, measurement of the muscle resistance to passive muscle movements, e.g., Ashworth Scale (AS), Modified Ashworth Scale (MAS) and Tardieu Scale, measurement of the muscle resistance to active muscle movement, speed of walking distances, electronic walking analysis and/or electronic gait analysis. Ashworth Scale, for instance, grades spasticity in a scale from 1 to 5: 1) no increase in muscle tone; 2) slight increase giving a catch when part is moved in flexion or extension; 3) more marked increase in tone but only after part is easily flexed; 4) considerable increase in tone; and 5) passive movement is difficult and affected part is rigid in flexion or extension. Spasticity in hip flexors, adductors, internal rotators, hamstrings, gastrocnemius are usually assessed. Ashworth scale is one of the most widely used methods of measuring spasticity, due in a large part to the simplicity and reproducible method.

The term “therapeutically effective amount” as used herein typically refers to a drug amount which, when administered to a subject, is sufficient to provide a therapeutic benefit, e.g. is sufficient for treating, preventing or delaying the progression of spasticity (e.g. the amount provides an amelioration of symptoms, e.g. it leads to a reduction in number and severity of seizures).

For the above-mentioned indications (the conditions and disorders) the appropriate dosage will vary depending upon, for example, the compound employed, the host, the mode of administration and the nature and severity of the condition being treated. However, in general, satisfactory results in animals are indicated to be obtained at a daily dosage of from about 0.01 to about 100 mg/kg body weight, preferably from about 1 to about 30 mg/kg body weight, e.g. 10 mg/kg. In larger mammals, for example humans, an indicated daily dosage is in the range from about 0.1 to about 1000 mg, preferably from about 1 to about 400 mg, most preferably from about 10 to about 100 mg of a 1H-quinazoline-2,4-dione of formula (I) conveniently administered, for example, in divided doses up to four times a day.

For use according to the invention, the 1H-quinazoline-2,4-diones of formula (I) may be administered as single active agent or in combination with other active agents, in any usual manner, e.g. orally, for example in the form of tablets, capsules or drinking solutions; rectally, for example in the form of suppositories; intravenous, for example in the form of injection solutions or suspensions; or transdermally, for example in the form of a patch.

In one embodiment, the manner of administration is oral administration, for example in the form of a tablet, capsule or drinking solution.

In one embodiment, the manner of administration is rectal administration, for example in the form of a suppository.

In one embodiment, the manner of administration is transdermal administration, for example in the form of a patch.

In one preferred embodiment, the manner of administration is oral administration.

Moreover, the present invention provides a pharmaceutical composition comprising a 1H-quinazoline-2,4-diones of formula (I) in association with at least one pharmaceutical carrier or diluent for the treatment, prevention or delay of progression of spasticity. Such compositions may be manufactured in conventional manner. Unit dosage forms may contain, for example, from about 2.5 to about 250 mg, preferably from about 2.5 to about 200 mg, more preferably from about 2.5 to about 100 mg, still more preferably from about 2.5 to about 50 mg and still more preferably from about 2.5 to about 25 mg, of one or more of the 1H-quinazoline-2,4-diones of formula (I).

The pharmaceutical compositions according to the invention are compositions for enteral administration, such as oral or rectal administration; or parenteral administration, such as intramuscular, intravenous, nasal or transdermal administration, to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

The pharmaceutical compositions of the present invention are prepared in a manner known per se, for example by means of conventional dissolving, lyophilizing, mixing, granulating or confectioning processes. Such processes are exemplified in WO 2005/079802, WO 2003/047581, WO 2004/000316, WO 2005/044265, WO 2005/044266, WO 2005/044267, WO 2006/114262 and WO 2007/071358.

Compositions for transdermal are described in Remington's Pharmaceutical Sciences 16^{th} Edition Mack; Sucker, Fuchs and Spieser, Pharmazeutische Technologie, 1^{st} Edition, Springer.

Efficacy of the compounds of the invention in the treatment of spasticity and related conditions may be demonstrated by any suitable in vitro or in vivo testing procedure. For example, the efficacy may be demonstrated using the following procedures.

### Oral Bioavailability of the Compounds of the Invention

Oral bioavailability of the compounds of the invention may be demonstrated using any generally known test in which the compound is administered orally and a biological effect observed.

Oral bioavailability of the compounds of the invention in the treatment of spasticity may be further quantified by the Maximal Electroshock test, which demonstrates that the compounds are orally bioavailable, penetrate the blood brain barrier and bind to the target receptor.

The oral bioavailability was tested using the audiogenic mouse test (Audiogenic seizures, R. L. Collins; Chapter 14, pages: 347-372. In: Experimental Models of Epilepsy; By: Pupura, Penry, Tower, Woodbury, Walter, Raven Press, New York, 1972. Standard Book Number: 0-911216-26-X) and/or the MES test. Where the MES test was used (as described below), the result is given in Table 1.

### Table 1: In-Vivo Activity of Parent Compounds and Prodrugs in the Murine Maximal Electro Shock Test

Compounds of the invention were tested in OF1 mice using the maximal electroshock test (MES Test) described in detail by Schmutz et al., Naunyn-Schmiedeberg's Arch Pharmacol 1990, 342, 61-66. Briefly, generalized tonic-clonic convulsions of the hind extremities were induced by passing electrical current through temporal electrodes (50 Hz, 18 mA, 0.2s). Mice treated by vehicle showed mean seizure durations of 12-14s. 30 mg/kg carbamazepine was used as a positive control; mice were classified as protected by a compound if the duration of the seizure lasted only 3 second or less. Five mice were used for each treatment condition and the percentage of protected mice was used as readout (i.e. a compound could give 0%, 20%, 40%, 60%, 80% or 100% protection). Compounds of the invention were given at a dose of 50 mg/kg, p.o., 1 hour prior to induction of convulsions (i.e. “pre-treatment time -1 h”). ED50 values (ED: effective dose) were calculated using GraphPad Prism, v4.02. 15 s after shock administration, mouse blood was collected for determination of compounds' blood exposure.

The results are shown below in Table 1.

TABLE 1

| | | In vivo | MES-Test | |
|---|---|---|---|---|
| | | orally | (1 h, po) | |
| Compound | Structure | active | ED50[mg/kg] | IUPAC name |
| A-1 | 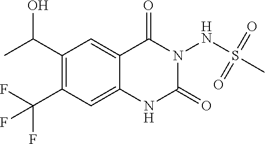 [SMILES: CC(O)c1cc2c(=O)n(NS(C)(=O)=O)c(=O)[nH]c2cc1C(F)(F)F] | Yes | 64 | N-[6-(1-Hydroxy-ethyl)-2,4-dioxo-7- trifluoromethyl-1,4-dihydro-2H-quinazolin-3-yl]- methanesulfonamide |
| A-2 | 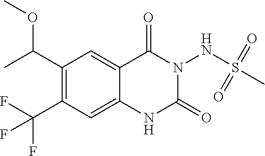 [SMILES: COC(C)c1cc2c(=O)n(NS(C)(=O)=O)c(=O)[nH]c2cc1C(F)(F)F] | Yes | 6.0 | N-[6-(1-Methoxy-ethyl)-2,4-dioxo-7- trifluoromethyl-1,4-dihydro-2H-quinazolin-3-yl]- methanesulfonamide |
| A-3 | 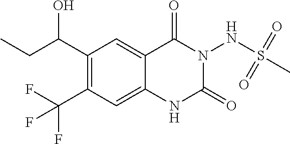 [SMILES: CCC(O)c1cc2c(=O)n(NS(C)(=O)=O)c(=O)[nH]c2cc1C(F)(F)F] | Yes | 19.6 | N-[6-(1-Hydroxy-propyl)-2,4-dioxo-7- trifluoromethyl-1,4-dihydro-2H-quinazolin-3-yl]- methanesulfonamide |
| A-4 | 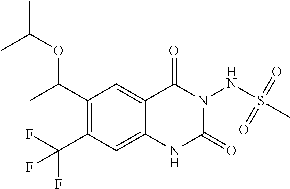 [SMILES: CC(C)OC(C)c1cc2c(=O)n(NS(C)(=O)=O)c(=O)[nH]c2cc1C(F)(F)F] | Yes | 15.6 | N-[6-(1-isopropoxy-ethyl)-2,4-dioxo-7- trifluoromethyl-1,4-dihydro-2H-quinazolin-3-yl]- methanesulfonamide |
| A-5 | 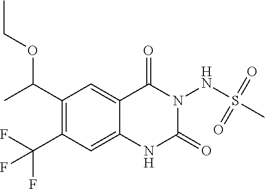 [SMILES: CCOC(C)c1cc2c(=O)n(NS(C)(=O)=O)c(=O)[nH]c2cc1C(F)(F)F] | Yes | 8.8 | N-[6-(1-Ethoxy-ethyl)-2,4-dioxo-7- trifluoromethyl-1,4-dihydro-2H-quinazolin-3-yl]- methanesulfonamide |
| A-6 | 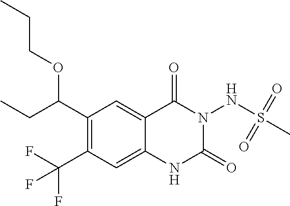 [SMILES: CCCOC(CC)c1cc2c(=O)n(NS(C)(=O)=O)c(=O)[nH]c2cc1C(F)(F)F] | Yes | nt¹ | N-[2,4-Dioxo-6-(1-propoxy-propyl)-7- trifluoromethyl-1,4-dihydro-2H-quinazolin-3-yl]- methanesulfonamide |
| A-7 | 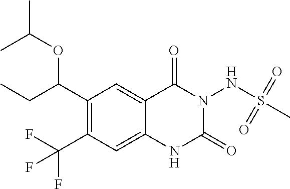 [SMILES: CCC(OC(C)C)c1cc2c(=O)n(NS(C)(=O)=O)c(=O)[nH]c2cc1C(F)(F)F] | Yes | 24.7 | N-[6-(1-isopropoxy-propyl)-2,4-dioxo-7- trifluoromethyl-1,4-dihydro-2H-quinazolin-3-yl]- methanesulfonamide |
| A-8 | 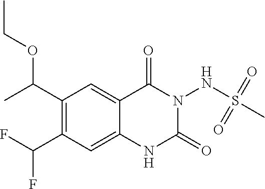 [SMILES: CCOC(C)c1cc2c(=O)n(NS(C)(=O)=O)c(=O)[nH]c2cc1C(F)F] | nt | nt | N-[7-Difluoromethyl-6-(1-ethoxy-ethyl)-2,4- dioxo-1,4-dihydro-2H-quinazolin-3-yl]- methanesulfonamide |
| A-9 | 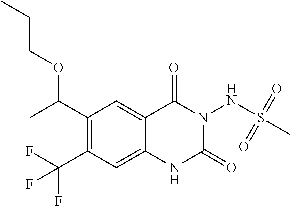 [SMILES: CCCOC(C)c1cc2c(=O)n(NS(C)(=O)=O)c(=O)[nH]c2cc1C(F)(F)F] | nt | nt | N-[2,4-Dioxo-6-(1-propoxy-ethyl)-7- trifluoromethyl-1,4-dihydro-2H-quinazolin-3-yl]- methanesulfonamide |
| A-10 | 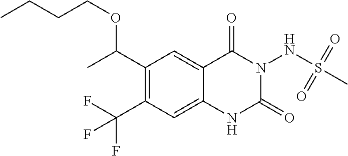 [SMILES: CCCCOC(C)c1cc2c(=O)n(NS(C)(=O)=O)c(=O)[nH]c2cc1C(F)(F)F] | nt | nt | N-[6-(1-Butoxy-ethyl)-2,4-dioxo-7- trifluoromethyl-1,4-dihydro-2H-quinazolin-3-yl]- methanesulfonamide |
| A-11 | 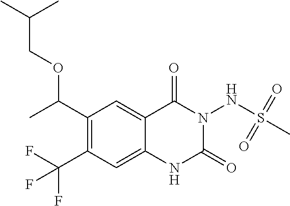 [SMILES: CC(C)COC(C)c1cc2c(=O)n(NS(C)(=O)=O)c(=O)[nH]c2cc1C(F)(F)F] | nt | nt | N-[6-(1-isobutoxy-ethyl)-2,4-dioxo-7- trifluoromethyl-1,4-dihydro-2H-quinazolin-3-yl]- methanesulfonamide |
| A-12 | 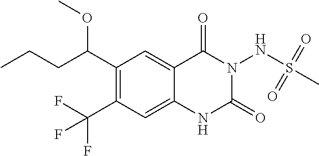 [SMILES: CCCC(OC)c1cc2c(=O)n(NS(C)(=O)=O)c(=O)[nH]c2cc1C(F)(F)F] | nt | nt | N-[6-(1-methoxy-butyl)-2,4-dioxo-7- trifluoromethyl-1,4-dihydro-2H-quinazolin-3-yl]- methanesulfonamide |
| A-13 | 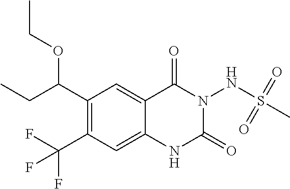 [SMILES: CCOC(CC)c1cc2c(=O)n(NS(C)(=O)=O)c(=O)[nH]c2cc1C(F)(F)F] | Yes | nt | N-[6-(1-Ethoxy-propyl)-2,4-dioxo-7- trifluoromethyl-1,4-dihydro-2H-quinazolin-3-yl]- methanesulfonamide |
| A-14 | 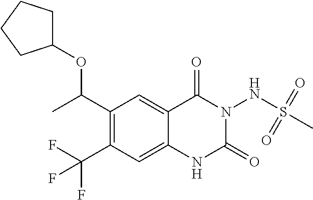 [SMILES: CC(OC1CCCC1)c1cc2c(=O)n(NS(C)(=O)=O)c(=O)[nH]c2cc1C(F)(F)F] | yes | nt | N-[6-(1-Cyclopentyloxy-ethyl)-2,4-dioxo-7- trifluoromethyl-1,4-dihydro-2H-quinazolin-3-yl]- methanesulfonamide |
| A-15 | 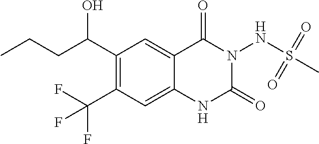 [SMILES: CCCC(O)c1cc2c(=O)n(NS(C)(=O)=O)c(=O)[nH]c2cc1C(F)(F)F] | Yes | 35 | N-[6-(1-Hydroxy-butyl)-2,4-dioxo-7- trifluoromethyl-1,4-dihydro-2H-quinazolin-3-yl]- methanesulfonamide |
| A-16 | 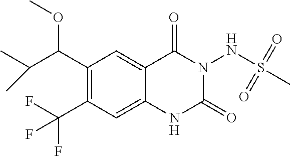 [SMILES: COC(c1cc2c(=O)n(NS(C)(=O)=O)c(=O)[nH]c2cc1C(F)(F)F)C(C)C] | nt | nt | N-[6-(1-Methoxy-2-methyl-propyl)-2,4-dioxo-7- trifluoromethyl-1,4-dihydro-2H-quinazolin-3-yl]- methanesulfonamide |
| A-17 | 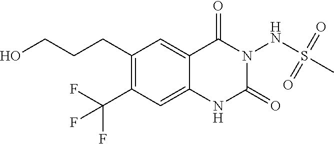 [SMILES: CS(=O)(=O)Nn1c(=O)[nH]c2cc(C(F)(F)F)c(CCCO)cc2c1=O] | nt | nt | N-[6-(3-Hydroxy-propyl)-2,4-dioxo-7- trifluoromethyl-1,4-dihydro-2H-quinazolin-3-yl]- methanesulfonamide |
| A-18 | 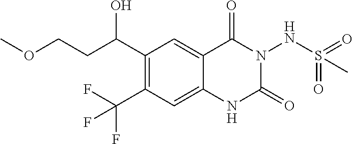 | Yes | nt | N-[6-(1-Hydroxy-3-methoxy-propyl)-2,4-dioxo- 7-trifluoromethyl-1,4-dihydro-2H-quinazolin-3- yl]-methanesulfonamide |
| A-19 | 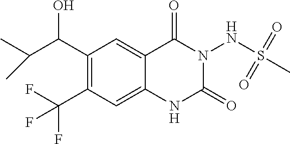 [SMILES: CC(C)C(O)c1cc2c(=O)n(NS(C)(=O)=O)c(=O)[nH]c2cc1C(F)(F)F] | nt | nt | N-[6-(1-Hydroxy-2-methyl-propyl)-2,4-dioxo-7- trifluoromethyl-1,4-dihydro-2H-quinazolin-3-yl]- methanesulfonamide |
| B-1 | 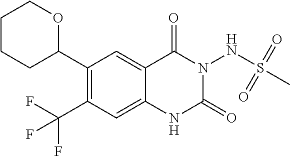 [SMILES: CS(=O)(=O)Nn1c(=O)[nH]c2cc(C(F)(F)F)c(C3CCCCO3)cc2c1=O] | nt | nt | N-[2,4-Dioxo-6-(tetrahydro-pyran-2-yl)-7- trifluoromethyl-1,4-dihydro-2H-quinazolin-3-yl]- methanesulfonamide |
| B-2 | 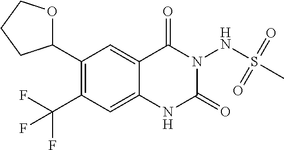 [SMILES: CS(=O)(=O)Nn1c(=O)[nH]c2cc(C(F)(F)F)c(C3CCCO3)cc2c1=O] | Yes | 12.8 (R)² 33.2 (S) | N-[2,4-Dioxo-6-(tetrahydro-furan-2-yl)-7- trifluoromethyl-1,4-dihydro-2H-quinazolin-3-yl]- methanesulfonamide |
| B-3 | 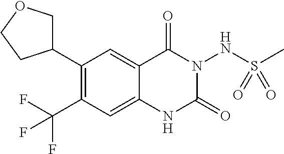 [SMILES: CS(=O)(=O)Nn1c(=O)[nH]c2cc(C(F)(F)F)c(C3CCOC3)cc2c1=O] | Yes | 20% @25³ | N-[2,4-Dioxo-6-(tetrahydro-furan-3-yl)-7- trifluoromethyl-1,4-dihydro-2 H-quinazolin-3-yl]-methanesulfonamide |
| C-1 | 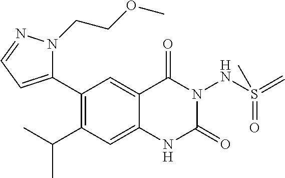 [SMILES: C=S(C)(=O)Nn1c(=O)[nH]c2cc(C(C)C)c(-c3ccnn3CCOC)cc2c1=O] | yes | 40% @25 | N-{7-isopropyl-6-[2-(2-methoxy-ethyl)-2H- pyrazol-3-yl]-2,4-dioxo-1,4-dihydro-2H- quinazolin-3-yl}-methanesulfonamide |
| C-2 | 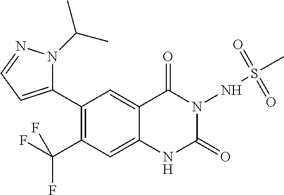 [SMILES: CC(C)n1nccc1-c1cc2c(=O)n(NS(C)(=O)=O)c(=O)[nH]c2cc1C(F)(F)F] | Yes | 17.7 | N-[6-(2-isopropyl-2H-pyrazol-3-yl)-2,4-dioxo-7- trifluoromethyl-1,4-dihydro-2H-quinazolin-3-yl]- methanesulfonamide |
| C-3 | 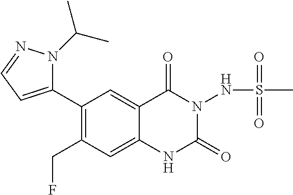 [SMILES: CC(C)n1nccc1-c1cc2c(=O)n(NS(C)(=O)=O)c(=O)[nH]c2cc1CF] | Yes | 13.5 | N-[7-Fluoromethyl-6-(2-isopropyl-2H-pyrazol-3- yl)-2,4-dioxo-1,4-dihydro-2H-quinazolin-3-yl]- methanesulfonamide |
| C-4 | 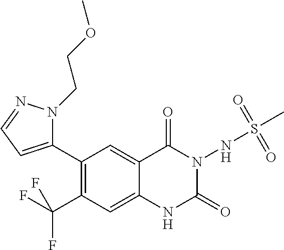 [SMILES: COCCn1nccc1-c1cc2c(=O)n(NS(C)(=O)=O)c(=O)[nH]c2cc1C(F)(F)F] | Yes | nt | N-{6-[2-(2-Methoxy-ethyl)-2H-pyrazol-3-yl]-2,4- dioxo-7-trifluoromethyl-1,4-dihydro-2H- quinazolin-3-yl}-methanesulfonamide |
| C-5 | 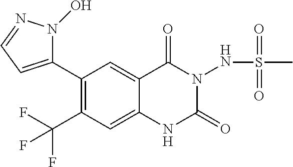 [SMILES: CS(=O)(=O)Nn1c(=O)[nH]c2cc(C(F)(F)F)c(-c3ccnn3O)cc2c1=O] | Yes | nt | N-[6-(2-Hydroxy-2H-pyrazol-3-yl)-2,4-dioxo-7- trifluoromethyl-1,4-dihydro-2H-quinazolin-3-yl]- methanesulfonamide |
| C-6 | 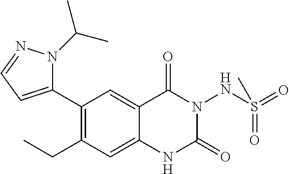 [SMILES: CCc1cc2[nH]c(=O)n(NS(C)(=O)=O)c(=O)c2cc1-c1ccnn1C(C)C] | yes | 20% @50 | N-[7-Ethyl-6-(2-isopropyl-2H-pyrazol-3-yl)-2,4- dioxo-1,4-dihydro-2H-quinazolin-3-yl]- methanesulfonamide |
| C-7 | 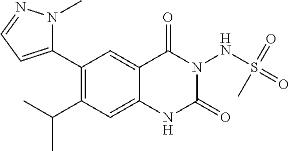 [SMILES: CC(C)c1cc2[nH]c(=O)n(NS(C)(=O)=O)c(=O)c2cc1-c1ccnn1C] | Yes | 6.9 | N-[7-Isopropyl-6-(2-methyl-2H-pyrazol-3-yl)- 2,4-dioxo-1,4-dihydro-2H-quinazolin-3-yl]- methanesulfonamide |
| C-8 | 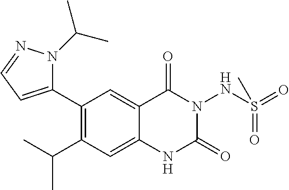 [SMILES: CC(C)c1cc2[nH]c(=O)n(NS(C)(=O)=O)c(=O)c2cc1-c1ccnn1C(C)C] | yes | 40% @50 | N-[7-Isopropyl-6-(2-isopropyl-2H-pyrazol-3-yl)- 2,4-dioxo-1,4-dihydro-2H-quinazolin-3-yl]- methanesulfonamide |
| C-9 | 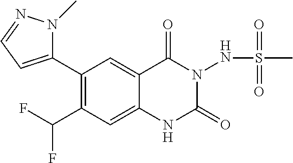 [SMILES: Cn1nccc1-c1cc2c(=O)n(NS(C)(=O)=O)c(=O)[nH]c2cc1C(F)F] | Yes | 7.5 | N-[7-Difluoromethyl-6-(2-methyl-2H-pyrazol-3- yl)-2,4-dioxo-1,4-dihydro-2H-quinazolin-3-yl]- methanesulfonamide |
| C-10 | 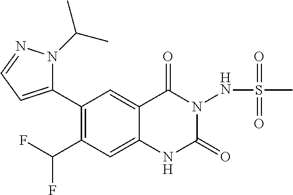 [SMILES: CC(C)n1nccc1-c1cc2c(=O)n(NS(C)(=O)=O)c(=O)[nH]c2cc1C(F)F] | Yes | 20.3 | N-[7-Difluoromethyl-6-(2-isopropyl-2H-pyrazol- 3-yl)-2,4-dioxo-1,4-dihydro-2H-quinazolin-3-yl]- methanesulfonamide |
| C-11 | 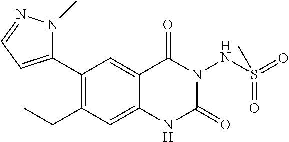 [SMILES: CCc1cc2[nH]c(=O)n(NS(C)(=O)=O)c(=O)c2cc1-c1ccnn1C] | Yes | 6.1 | N-[7-Ethyl-6-(2-methyl-2H-pyrazol-3-yl)-2,4- dioxo-1,4-dihydro-2H-quinazolin-3-yl]- methanesulfonamide |
| C-12 | 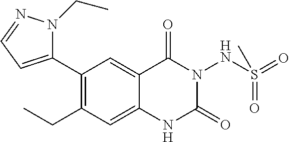 [SMILES: CCc1cc2[nH]c(=O)n(NS(C)(=O)=O)c(=O)c2cc1-c1ccnn1CC] | Yes | 42.8 | N-[7-Ethyl-6-(2-ethyl-2H-pyrazol-3-yl)-2,4- dioxo-1,4-dihydro-2H-quinazolin-3-yl]- methanesulfonamide |
| C-13 | 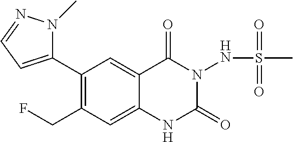 [SMILES: Cn1nccc1-c1cc2c(=O)n(NS(C)(=O)=O)c(=O)[nH]c2cc1CF] | nt | nt | N-[7-Fluoromethyl-6-(2-methyl-2H-pyrazol-3- yl)-2,4-dioxo-1,4-dihydro-2H-quinazolin-3-yl]- methanesulfonamide |
| C-14 | 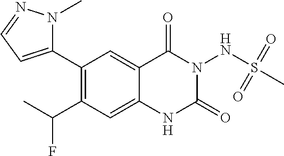 [SMILES: CC(F)c1cc2[nH]c(=O)n(NS(C)(=O)=O)c(=O)c2cc1-c1ccnn1C] | nt | nt | N-[7-(1-fluoro-ethyl)-6-(2-methyl-2H-pyrazol-3- yl)-2,4-dioxo-1,4-dihydro-2H-quinazolin-3-yl]- methanesulfonamide |
| C-15 | 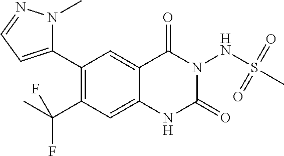 [SMILES: Cn1nccc1-c1cc2c(=O)n(NS(C)(=O)=O)c(=O)[nH]c2cc1C(C)(F)F] | Yes | 80% @20 | N-[7-(1,1-difluoro-ethyl)-6-(2-methyl-2H- pyrazol-3-yl)-2,4-dioxo-1,4-dihydro-2H- quinazolin-3-yl]-methanesulfonamide |
| C-16 | 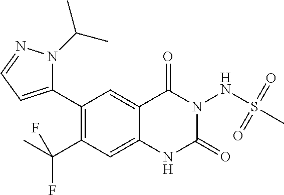 [SMILES: CC(C)n1nccc1-c1cc2c(=O)n(NS(C)(=O)=O)c(=O)[nH]c2cc1C(C)(F)F] | Yes | nt | N-[7-(1,1-difluoro-ethyl)-6-(2-isopropyl-2H- pyrazol-3-yl)-2,4-dioxo-1,4-dihydro-2H- quinazolin-3-yl]-methanesulfonamide |
| C-17 | 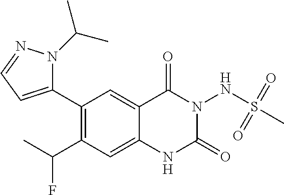 [SMILES: CC(F)c1cc2[nH]c(=O)n(NS(C)(=O)=O)c(=O)c2cc1-c1ccnn1C(C)C] | Yes | >20 | N-[7-(1-fluoro-ethyl)-6-(2-isopropyl-2H-pyrazol- 3-yl)-2,4-dioxo-1,4-dihydro-2H-quinazolin-3-yl]- methanesulfonamide |
| C-18 | 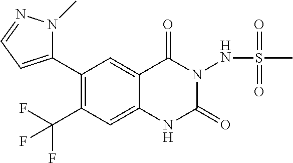 [SMILES: Cn1nccc1-c1cc2c(=O)n(NS(C)(=O)=O)c(=O)[nH]c2cc1C(F)(F)F] | Yes | 14.8 | N-[6-(2-Methyl-2H-pyrazol-3-yl)-2,4-dioxo-7- trifluoromethyl-1,4-dihydro-2H-quinazolin-3-yl]- methanesulfonamide |
| Comparative | 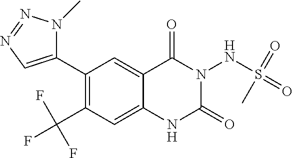 [SMILES: Cn1nncc1-c1cc2c(=O)n(NS(C)(=O)=O)c(=O)[nH]c2cc1C(F)(F)F] | No | 0% @50 | N-(6-(1-methyl-1H-1,2,3-triazol-5-yl)-2,4-dioxo- 7-trifluoromethyl-1,4-dihydro-2H-quinazolin-3- yl]-methanesulfonamide |

¹The term “nt” through out the table means “not tested”
²(R) and (S) indicate the two enantiomers.
³The term “20% @25” means 20% protection at 50 mg/kg.

This data shows that the compounds for use in the invention exhibit beneficial oral bioavallability relating to the comparative example (not in accordance with the invention).

Animal Models for Spasticity Astrocytes may be isolated from lumbar spinal cord of postnatal day 0-1 rat pups, using Papain Dissection System. Thereafter the cells may be cultured with DMEM, supplied with 10% fetal calf serum. To purify astrocytes, mechanical shaking may be used and cells were re-fed with fresh DMEM-10% FCS every three days until confluent and then passed into 24-well plates. On the day of the release experiment the medium may be replaced by 300 μl/well artificial CSF (bubbled with 95% O2/5% CO2; pH adjusted to 7.3). After 10 minutes in the incubator the cells may be stimulated with AMPA (1, 10 or 30 μl) in the presence or absence of different concentrations of AMPA antagonist. Samples may be analyzed for glutamate by HPLC.

In rats, a reflex response similar to the human H-reflex can be elicited by low intensity electrical stimulation of the tibial nerve. This low intensity electrical stimulation activates primary muscle spindle afferents which through a monosynaptic reflex via the lumbar spinal cord causes excitation of spinal α-motoneurons. The excitation of the motoneurons is quantified as increase in the electro-myogram (EMG). Reduction of the amplitude of the H-reflex is a read-out for anti-spastic activity. For recording of H-reflexes, the rat was anesthetized with pentobarbital, left hindlimb was denervated from all nerves except the tibial nerve. The tibial nerve was exposed and mounted on bipolar platinum electrodes for stimulation (single square-wave shocks, 0.2 ms duration at 1.4-1.6 times the reflex threshold). EMG recordings were made with a pair of skin clip surface electrodes from the plantar food muscles.

Human methods to quantify spasticity after administration of the pharmaceutical composition containing 1H-quinazoline-2,4-diones spasticity may be assessed in various manner. The assessment may be done at specific time points after the administration of the pharmaceutical composition containing 1H-quinazoline-2,4-diones. Spasticity may be assessed and measured as described hereinafter.

### Clinical Examination

By the clinical examination strength and reflexes may both be assessed in this examination. The clinician asks the patient to relax and then moves the joints through their full range of motion at various speeds. Spastic muscles may have a “spastic catch,” exhibit the “clasped knife” phenomenon, or both. Observing the person with spasticity perform activities such as walking, drinking from an open cup, and moving from one position to another often yields valuable information. The clinical examination also includes an evaluation of deep tendon reflexes. The most commonly used method of testing these reflexes is the tapping technique. With the patient sitting on the examination table and his or her legs hanging freely, the examiner gently but firmly taps below the knee (testing the patellar reflex), first on one leg and then the other. The responses should be the same in the two legs. Similar techniques may be used to test reflexes in the Achilles tendon (behind the ankle), and reflexes may also be checked in the biceps, triceps, and brachioradialis muscles of the arms.

### Rating Scale

Rating scales may be used to measure spasticity and the response to treatment. Both the original and modified versions of the Ashworth Scale may be used for measuring the treatment response. Another scale that may be used in measuring response to treatment is a spasm scale. This scale simply requires the assessor to count the number of spasms that the patient has in a set period of time, typically one hour.

Evaluation of the Capacity of Glutamate (AMPA) Receptor Competitive Antagonist 1H-guinazoline-2,4-diones of Formula C7 to Inhibit Experimental Spasticity

The study is used to examine the influence of (a) Compound-AMPA antagonist of formula (I), e.g. compound C7, (b) vehicle and/or (c) NBQX (ICN Chemical supply).

### Methods

### Compounds

Compounds of formula (I), e.g. compound C7, may be orally administered.

NBQX may be injected subcutaneously.

### Animals

Biozzi ABH mice.

### Induction of Experimental Autoimmune Encephalomyelitis

6-8 week mice may be injected subcutaneously in the flank with 1mg of freeze-dried mouse spinal cord homogenate (SCH) emulsified in Freunds adjuvant containing 60 μg Mycobacterium tuberculosis H37Ra and M. butyicum on day 0 and day 7 [Baker et al., J. Neuroimmunol. 1990; 28:261-270].

Animals shall be weighed and may be scored daily from day 11 onwards according to the criteria below. On about day 13 post-inoculation (p.i.), mice typically will have lost more than 1.5g overnight. Weight loss typically continues for a few days. On about day 15 clinical signs started with ascending paralysis from the tail. This is scored:

Normal tail=0. Fully flaccid tail=1, which corresponds to a completely paralysed tail. If the tail does not lift but has some tone, e.g. the tail can bend round finger or the tail rotates when the mouse is lifted by the scruff of the neck=0.5. This may be the typical score of remission 1.

Impaired righting reflex.=2, which means that when the animal is turned on back it does not right itself, If the mouse rights itself slowly it gets a score of=1.5. Hindlimb paresis=3, which correspond to a significant loss of motor function of the hindlimbs. Hindlimb gait disturbance=2.5. The score 2-3 may be the typical score of remission. Complete hindlimb paralysis=4, which means that both hind limbs drag. Limbs virtually paralysed but have some minor movement or one leg fully paralysed=3.5. Moribund/Death=5. If forelimbs become paralysed the animal is euthanized. A weight loss limit of about 35% from the day 11 weight has been set [O'Neill et al., Journal of Neuroimmunology, Vol. 33, Issue 1, 1991, 37-42]. Relapse=Increase of Disease Score, usually accompanied with weight loss.

The data may be presented as the mean daily clinical score±standard error of the mean (SEM) or the mean maximal clinical score of the group (Group Score)±SEM; the mean maximal clinical score of the animals that developed clinical disease (EAE Score)±SEM and the mean day of onset±standard deviation (SD). Differences between groups were assessed using non-parametric, Mann Whitney U statistics using Sigmastat Software.

### Induction and Inhibition of Spasticity

Experimental Allergic/Autoimmune encephalitis (EAE) may be induced in 50-100 Biozzi ABH mice. These were monitored daily from day 11 onwards for the development of EAE and visually assessed for the development of hindlimb spasticity. This typically occurs after 3-4 clinical attacks and is developed in about 50% of immunized mice within 4-8 months [Baker et al., Nature 2000, 404:84-87]. This assay is responsive to cannabinoid receptor agonist or GABA-receptor agonists (baclofen) and does not respond to water (following oral treatment), saline, intralipid, dimethyl sulphoxidetethanot-cremophor-phosphate buffered saline (1:1:18) or Klucel™ vehicles.

Spasticity may be assessed during remission from active paralytic episodes by the force required to bend the hind limb to full flexion against a strain gauge [Baker et al, Nature 2000, 404:84-87]. The limb, may be extended two-three times and then gently pressed against a strain gauge to full flexion. The measurement of left then right hindlimbs may be repeated typically 5 times per time point. Analogue signals may be amplified, digitized and captured for computer analyses under Windows™. The data may be analyzed and a mean score for each limb at each time point calculated while forces may be converted to Newtons. Each group typibally contains a minimum of 5 different animals, typically 7-8 mice/group and the results represent the mean SEM resistance to flexion force (N) or individual limbs, which were compared using repeated measures/analysis of variance or paired t tests using statistics software.

Initial assessment may be following the oral administration of compound of formula C7 (25-50 mg/kg). Spasticity may be assessed at baseline 10, 30, 60 and 90min following treatment. The vehicle e.g., Klucel, is of inert activity in this assay. A dose-response down to inactive doses may be performed. The drugs may be active within 25-60 minutes following administration. To allow for a direct comparison of doses to be made, these assays may be performed in the same animals following at least one week wash-out. Important observations may be repeated in additional drug-naïve cohorts of animals. As comparator, the literature AMPA antagonist NBQX, may be administered (i.p. or sc). To assess receptor tolerance to test drug, spasticity may be measured at baseline, 30 min, 60 min, 120 min and 24 hours and repeated on day 7).

1. A compound of formula (I); 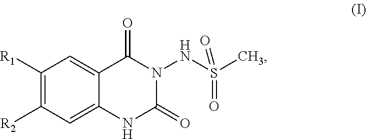
wherein
R₁ is C₁-C₆alkyl substituted by one, two or three substituents selected from hydroxy, C₁-C₆alkoxy or C₅-C₆cycloalkoxy; C₅-C₆cycloalkyl substituted by one, two or three substituents selected from hydroxy, C₁-C₆alkoxy or C₅-C₆cycloalkoxy; or
R₁ is 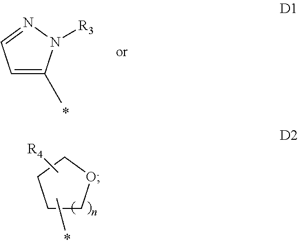
R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃fluoroalkyl;
and their pharmaceutically acceptable salts;
for use in a method for the treatment, prevention or delay of progression of spasticity.

2. A compound of formula (I) according to claim 1, wherein R₁ is D1 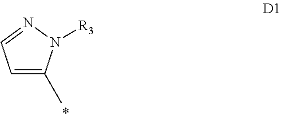
R₃ is C₁-C₆alkyl, hydroxy or C₁-C₆alkoxy-C₁-C₆alkyl;
R₂ is C₁-C₃alkyl or C₁-C₃fluoroalkyl;
and their pharmaceutically acceptable salts;
for use in a method for the treatment, prevention or delay of progression of spasticity.

3. A compound of formula (I) according to claim 1, wherein R₁ is D2 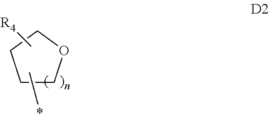
R₄ is hydrogen or C₁-C₆alkyl;
n is 1 or 2;
R₂ is C₁-C₃alkyl or C₁-C₃fluoroalkyl;
and their pharmaceutically acceptable salts;
for use in a method for the treatment, prevention or delay of progression of spasticity.

4. A compound of formula (I) according to claim 1, selected from the group consisting of:
N-[6-(1-Hydroxy-ethyl)-2,4-dioxo-7-trifluoromethyl-1,4-dihydro-2H-quinazolin-3-yl]-methanesulfonamide;
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[6-Dioxo-6-(1-propoxy-propyl)-7-trifluoromethyl-1,4-dihydro-2H-quinazolin-3-yl]-methanesulfonamide;
N-[6-(1-isopropoxy-propyl)-2,4-dioxo-7-trifluoromethyl-1,4-dihydro-2H-quinazolin-3-yl]-methanesulfonamide;
N-[7-Difluoromethyl-6-(1-ethoxy-ethyl)-2,4-dioxo-1,4-dihydro-2H-quinazolin-3-yl]-methanesulfonamide;
N-[2,4-Dioxo-6-(1-propoxy-ethyl)-7-trifluoromethyl-1,4-dihydro-2H-quinazolin-3-yl]-methanesulfonamide;
N-[6-(1-Butoxy-ethyl)-2,4-dioxo-7-trifluoromethyl-1,4-dihydro-2H-quinazolin-3-yl]-methanesulfonamide;
N-[6-(1-Isobutoxy-ethyl)-2,4-dioxo-7-trifluoromethyl-4-dihydro-2H-quinazolin-3-yl]-methanesulfonamide;
N-[6-(1-methoxy-butyl)-2,4-dioxo-7-trifluoromethyl-1,4-dihydro-2H-quinazolin-3-yl]-methanesulfonamide;
N-[6-(1-Ethoxy-propyl)-2,4-dioxo-7-trifluoromethyl-1,4-dihydro-2H-quinazolin-3-yl]-methanesulfonamide;
N-[6-(1-Cyclopentyloxy-ethyl)-2,4-dioxo-7-trifluoromethyl-1,4-dihydro-2H-quinazolin-3-yl]-methanesulfonamide;
N-[6-(1-Hydroxy-butyl)-2,4-dioxo-7-trifluoromethyl-1,4-dihydro-2H-quinazolin-3-yl]-methanesulfonamide;
N-[6-(1-Methoxy-2-methyl-propyl)-2,4-dioxo-7-trifluoromethyl-1,4-dihydro-2H-quinazolin-3-yl]-methanesulfonamide;
N-[6-(3-Hydroxy-propyl)-2,4-dioxo-7-trifluoromethyl-1,4-dihydro-2H-quinazolin-3-yl]-methanesulfonamide;
N-[6-(1-Hydroxy-3-methoxy-propyl)-2,4-dioxo-7-trifluoromethyl-1,4-dihydro-2H-quinazolin-3-yl]-methanesulfonamide;
N-[6-(1-Hydroxy-2-methyl-propyl)-2,4-dioxo-7-trifluoromethyl-1,4-dihydro-2H-quinazolin-3-yl]-methanesulfonamide;
N-[2,4-Dioxo-6-(tetrahydro-pyran-2-yl)-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N[2,4-Dioxo-6-(tetrahydro-furan-3-yl)-7-trifluoromethyl-1,4-dihydro-2H-quinazolin-3-yl]-methanesulfonamide;
N-{7-Isopropyl-6-[2-(2-methoxy-ethyl)-2H-pyrazol-3-yl]-2,4-dioxo-1,4-dihydro-2H-quinazolin-3-yl}-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N[7-Fluoromethyl-6-(2-isopropyl-2H-pyrazol-3-yl)-2,4-dioxo-1,4-dihydro-2H-quinazolin-3-yl]-methanesulfonamide;
N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;
N-[6-(2-Hydroxy-2H-pyrazol-3-yl)-2,4-dioxo-7-trifluoromethyl-1,4-dihydro-2H-quinazolin-3-yl]-methanesulfonamide;
N-[7-Ethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-isopropyl-21-1-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-ethyl-2H-pyrazol-3-yl)-2,4-dioxo-1,4-dihydro-2H-quinazolin=3-yl]-methanesulfonamide;
N-[7-Fluoromethyl-6-(2-methyl-2H-pyrazol-3-yl)-2,4-dioxo-1,4-dihydro-2H-quinazolin-3-yl]-methanesulfonamide;
N-[7-(1-fluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-isopropyl-2H-pyrazol-3-yl)-2,4-dioxo-1,4-dihydro-2H-quinazolin-3-yl]-methanesulfonamide;
N-[7-(1-fluoro-ethyl)-6-(2-isopropyl-2H-pyrazol-3-yl)-2,4-dioxo-1,4-dihydro-2H-quinazolin-3-yl]-methanesulfonamide; or
N-[6-(2-Methyl-2H-pyrazol-3-yl)-2,4-dioxo-7-trifluoromethyl-1,4-dihydro-2H-quinazolin-3-yl]-methanesulfonamide.

5. A compound of formula (I) according to claim 1 selected from the group consisting of:
N-[6-(1-Hydroxy-ethyl)-2,4-dioxo-7-trifluoromethyl-1,4-dihydro-2H-quinazolin-3-yl]-methanesulfonamide;
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[2,4-Dioxo-6-(1-propoxy-propyl)-7-trifluoromethyl-1,4-dihydro-2H-quinazolin-3-yl]-methanesulfonamide;
N-[6-(1-isopropoxy-propyl)-2,4-dioxo-7-trifluoromethyl-1,4-dihydro-2H-quinazolin-3-yl]-methanesulfonamide;
N-[6-(1-Cyclopentyloxy-ethyl)-2,4-dioxo-7-trifluoromethyl-1,4-dihydro-2H-quinazolin-3-yl]-methanesulfonamide
N-[6-(1-Hydroxy-butyl)-2,4-dioxo-7-trifluoromethyl-1,4-dihydro-2H-quinazolin-3-yl]-methanesulfonamide;
N-[6-(1-Hydroxy-3-methoxy-propyl)-2,4-dioxo-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[2,4-Dioxo-6-(tetrahydro-furan-3-yl)-7-trifluoromethyl-1,4-dihydro-2H-quinazolin-3-yl]-methanesulfonamide;
N-{7-Isopropyl-6-[2-(2-methoxy-ethyl)-2H-pyrazol-3-yl]-2,4-dioxo-1,4-dihydro-2H-quinazolin-3-yl}-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;
N-[6-(2-Hydroxy-2H-pyrazol-3-yl)-2,4-dioxo-7-trifluoromethyl-1,4-dihydro-2H-quinazolin-3-yl]-methanesulfonamide;
N-[7-Ethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-isopropyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-ethyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-isopropyl-2H-pyrazol-3-yl)-2,4-dioxo-1,4-dihydro-2H-quinazolin-3-yl]-methanesulfonamide
N-[7-(1-fluoro-ethyl)-6-(2-isopropyl-2H-pyrazol-3-yl)-2,4-dioxo-1,4-dihydro-2H-quinazolin-3-yl]-methanesulfonamide; and
N-[6-(2-Methyl-2H-pyrazol-3-yl)-2,4-dioxo-7-trifluoromethyl-1,4-dihydro-2H-quinazolin-3-yl]-methanesulfonamide.

6. A compound of formula (I) according to claim 1, selected from the group consisting of:
N-[6-(1-Hydroxy-ethyl)-2,4-dioxo-7-trifluoromethyl-1,4-dihydro-2H-quinazolin-3-yl]-methanesulfonamide;
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[6-(1-isopropoxy-propyl)-2,4-dioxo-7-trifluoromethyl-1,4-dihydro-2H-quinazolin-3-yl]-methanesulfonamide;
N-[6-(1-Hydroxy-butyl)-2,4-dioxo-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[2,4-Dioxo-6-(tetrahydro-furan-3-yl)-7-trifluoromethyl-1,4-dihydro-2H-quinazolin-3-yl]-methanesulfonamide;
N-{7-Isopropyl-6-[2-(2-methoxy-ethyl)-2H-pyrazol-3-yl]-2,4-dioxo-1,4-dihydro-2H-quinazolin-3-yl}-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-isopropyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-ethyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[7-(1-fluoro-ethyl)-6-(2-isopropyl-2H-pyrazol-3-yl)-2,4-dioxo-1,4-dihydro-2H-quinazolin-3-yl]-methanesulfonamide; and
N-[6-(2-Methyl-2H-pyrazol-3-yl)-2,4-dioxo-7-trifluoromethyl-1,4-dihydro-2H-quinazolin-3-yl]-methanesulfonamide.

7. A compound of formula (I) according to claim 1, selected from the group consisting of:
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[6-(2-Methyl-2H-pyrazol-3-yl)-2,4-dioxo-7-trifluoromethyl-1,4-dihydro-2H-quinazolin=3-yl]-methanesulfonamide;

8. A compound of formula (I) according to claim 1, selected from the group consisting of:
N-[6-(1-Methoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide.

9. A compound of formula (I) according to claim 1, wherein the compound of formula (I) is N-[7-Isopropyl-6-(2-methyl-2H-pyrazol-3-yl)-2,4-dioxo-1,4-dihydro-2H-quinazolin-3-yl]-methanesulfonamide;

10. A compound of formula (I) according to claim 1, wherein the compound of formula (I) is N-[6-(1-Methoxy-ethyl)-2,4-dioxo-7-trifluoromethyl-1,4-dihydro-2H-quinazolin-3-yl]-methanesulfonamide.

11. A compound of formula (I) according to claim 1, wherein the compound of formula (I) is N-[6-(1-Ethoxy-ethyl)-2,4-dioxo-7-trifluoromethyl-1,4-dihydro-2H-quinazolin-3-yl]-methanesulfonamide.

12. A compound of formula (I) according to claim 1, wherein the compound of formula (I) is N-[7-Ethyl-6-(2-methyl-2H-pyrazol-3-yl)-2,4-dioxo-1,4-dihydro-2H-quinazolin-3-yl]-methanesulfonamide.

13. Use of a compound of formula (I) as defined in anyone of claim 1, in the manufacture of a medicament for the treatment, prevention or delay of progression of spasticity.

14. A method for the treatment, prevention or delay of progression of spasticity in a subject in need of such treatment, which comprises administering to said subject a therapeutically effective amount of a compound of formula (I) as defined in claim 1.
